(19) United States
(12) Patent Application Publication
(10) Pub. No.: US 2008/0027456 A1
(43) Pub. Date: Jan. 31, 2008

(54) Bone treatment systems and methods

(76) Inventors: Csaba Truckai, Saratoga, CA (US); Robert Luzzi, Pleasanton, CA (US); John H. Shadduck, Tiburon, CA (US); Eric Buehlmann, Redwood City, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/880,475

(22) Filed: Jul. 19, 2007

Related U.S. Application Data
(60) Provisional application No. 60/831,664, filed on Jul. 19, 2006.

(51) Int. Cl. A61B 17/58

(52) U.S. Cl. 606/94

(57) ABSTRACT

A bone cement injector used for treating vertebral compression fractures has diamond-like coatings or amorphous carbon based coatings with a high hardness and a low coefficient of friction on an interior flow channel thereof. Such a bone cement injector includes a sensor system for sensing retrograde bone cement flows that can migrate along a fractured path toward a pedicle and risk leakage into the spinal canal. An energy delivery system can be coupled to the injector for applying energy to tissue and/or to bone cement that migrates in a retrograde direction, wherein the energy polymerizes the cement and/or coagulates tissue to inhibit further retrograde cement migration.

### CROSS-REFERENCE TO RELATED APPLICATIONS

This application claims the benefit of U.S. Provisional Patent Application No. 60/831,664 filed Jul. 19, 2006, the entire contents of which are incorporated herein by reference and should be considered a part of this specification. This application is related to the following U.S. patent applications: Ser. No. 11/165,652 filed Jun. 24, 2005; Ser. No. 11/165,651 filed Jun. 24, 2005; U.S. patent application Ser. No. 11/208,448 filed Aug. 20, 2005; No. 60/713,521 filed Sep. 1, 2005; Ser. No. 11/209,035 filed Aug. 22, 2005. The entire contents of all of the above applications are hereby incorporated by reference and should be considered a part of this specification.

### BACKGROUND OF THE INVENTION

1. Field of the Invention

The present invention relates in certain embodiments to medical devices for treating osteoplasty procedures such as vertebral compression fractures. More particularly, embodiments of the invention relate to instruments and methods for controlling bone cement flows and viscosity into the interior of a vertebra, including an injector having a lubricious inner surface that defines a flow channel through the injector, the lubricious surface facilitating the flow of bone fill material through the introducer.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80-year-old women. Medical advances aimed at slowing or arresting bone loss from aging have not provided solutions to this problem. Further, the population affected will grow steadily as life expectancy increases. Osteoporosis affects the entire skeleton but most commonly causes fractures in the spine and hip. Spinal or vertebral fractures also cause other serious side effects, with patients suffering from loss of height, deformity and persistent pain which can significantly impair mobility and quality of life. Fracture pain usually lasts 4 to 6 weeks, with intense pain at the fracture site. Chronic pain often occurs when one vertebral level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of with collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporosis bone, the sponge-like cancellous bone has pores or voids that increase in dimension making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. Vertebroplasty is the percutaneous injection of PMMA (polymethylmethacrylate) into a fractured vertebral body via a trocar and cannula. The targeted vertebrae are identified under fluoroscopy. A needle is introduced into the vertebrae body under fluoroscopic control, to allow direct visualization. A bilateral transpedicular (through the pedicle of the vertebrae) approach is typical but the procedure can be done unilaterally. The bilateral transpedicular approach allows for more uniform PMMA infill of the vertebra.

In a bilateral approach, approximately 1 to 4 ml of PMMA is used on each side of the vertebra. Since the PMMA needs to be is forced into the cancellous bone, the techniques require high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasation are critical to the technique—and the physician terminates PMMA injection when leakage is evident. The cement is injected using syringes to allow the physician manual control of injection pressure.

Kyphoplasty is a modification of percutaneous vertebroplasty. Kyphoplasty involves a preliminary step consisting of the percutaneous placement of an inflatable balloon tamp in the vertebral body. Inflation of the balloon creates a cavity in the bone prior to cement injection. The proponents of percutaneous kyphoplasty have suggested that high pressure balloon-tamp inflation can at least partially restore vertebral body height. In kyphoplasty, some physicians state that PMMA can be injected at a lower pressure into the collapsed vertebra since a cavity exists, when compared to conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles.

Leakage of PMMA during vertebroplasty can result in very serious complications including compression of adjacent structures that necessitate emergency decompressive surgery. See “Anatomical and Pathological Considerations in Percutaneous Vertebroplasty and Kyphoplasty: A Reappraisal of the Vertebral Venous System”, Groen, R. et al, Spine Vol. 29, No. 13, pp 1465-1471 2004. Leakage or extravasation of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (May 2004) pp. 478-82, (http://www.jkns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. 2004 February; 25(2):175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al, “Asymptomatic diffuse pulmonary embolism caused by acrylic cement: an unusual complication of percutaneous vertebroplasty”, Ann. Rheum. Dis. 2003;62:85-86. The vapors from PMMA preparation and injection also are cause for concern. See Kirby, B, et al., “Acute bronchospasm due to exposure to polymethylmethacrylate vapors during percutaneous vertebroplasty”, Am. J. Roentgenol. 2003; 180:543-544.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon applies also compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which crushes and compacts cancellous bone. Expansion of the balloon under high pressures close to cortical bone can fracture the cortical bone, typically the endplates, which can cause regional damage to the cortical bone with the risk of cortical bone necrosis. Such cortical bone damage is highly undesirable as the endplate and adjacent structures provide nutrients for the disc.

Kyphoplasty also does not provide a distraction mechanism capable of 100% vertebral height restoration. Further, the kyphoplasty balloons under very high pressure typically apply forces to vertebral endplates within a central region of the cortical bone that may be weak, rather than distributing forces over the endplate.

There is a general need to provide bone cements and methods for use in treatment of vertebral compression fractures that provide a greater degree of control over introduction of cement and that provide better outcomes.

### SUMMARY OF THE INVENTION

Certain embodiments of the invention provide vertebroplasty systems and methods for sensing retrograde bone cement flows that can migrate along a fractured path toward a pedicle and risk leakage into the spinal canal. The physician can be alerted instantaneously of cement migration in a direction that can impinge on nerves or the spinal cord. Other embodiments include integrated sensing systems and energy delivery systems for applying energy to tissue and/or to bone cement that migrates in a retrograde direction wherein the energy polymerizes the cement and/or coagulates tissue to create a dam to prevent further cement migration. In another embodiment, the systems provide a cooling system for cooling bone cement in a remote container or injection cannula for controlling and extending the working time of a bone cement. In another embodiment, the bone cement injection system includes a thermal energy emitter for warming a chilled bone cement in an injector or for applying sufficient energy to accelerate polymerization and thereby increase the viscosity of the bone cement.

In one embodiment, a computer controller is provided to control cement inflow parameters from, for example, a hydraulic source, the sensing system and energy delivery system parameters for selectively heating tissue or polymerizing cement at both the interior and exterior of the injector to thereby control all parameters of cement injection to reduce workload on the physician.

In certain embodiments, a lubricous surface layer is provided in the flow passageway of the bone cement injector to prevent sticking particularly when heating the cement.

In accordance with one embodiment, an apparatus for delivering a bone fill material to a vertebra is provided. The apparatus comprises an injector configured for introduction into a vertebral body, at least a portion of the injector positionable within the vertebral body, the injector having a lubricious surface layer that defines a flow channel extending through the injector to at least one outlet opening. The apparatus also comprises a thermal energy emitter operably coupled to the introducer, at least a portion of the surface layer disposed between the thermal energy emitter and the flow channel, the thermal energy emitter configured to apply energy to the bone fill material flowing through the flow channel via conduction through the surface layer.

In accordance with another embodiment, an apparatus for delivering a bone cement to a bone, comprising a bone cement injector having a flow channel extending therethrough to at least one outlet opening in a distal end of the injector, wherein a surface of the flow channel comprises a polymeric layer.

In accordance with yet another embodiment, an apparatus for delivering a bone cement to a bone, comprising a bone cement injector having a flow channel extending therethrough to at least one outlet opening in a distal end of the injector, wherein a surface of the flow channel comprises a ceramic layer.

### BRIEF DESCRIPTION OF THE DRAWINGS

These and other features, aspects and advantages of the present inventions will now be described in connection with preferred embodiments, in reference to the accompanying drawings. The illustrated embodiments, however, are merely examples and are not intended to limit the inventions. The drawings include the following 8 figures.

FIG. 1 is a schematic perspective view of a bone cement injection system and sensing system in accordance with one embodiment.

FIG. 2 is another schematic view of the bone cement injector of FIG. 1.

FIG. 3A is a schematic sectional view of a vertebra showing a first step in a bone cement injection method according to one embodiment.

FIG. 3B is a schematic sectional view of the vertebra of FIG. 3A showing a subsequent step in a bone cement injection method.

FIG. 3C is a schematic sectional view similar to FIGS. 3A-3B showing a subsequent step in a bone cement injection method wherein a sensing system detects a retrograde flow.

FIG. 4 is a schematic view of the bone cement injector of FIGS. 1-2.

FIG. 5 is a schematic cross-sectional view of a distal portion of the bone cement injector of FIGS. 1-2 with a thermal energy emitter in an interior bore of the injector.

FIG. 6 is a schematic cross-sectional view of a distal portion of another embodiment of an injector having a lubricious surface layer or coating in the interior bore of the injector.

### DETAILED DESCRIPTION OF THE PREFERRED EMBODIMENT

Definitions

“Bone fill, fill material, or infill material or composition” includes its ordinary meaning and is defined as any material for infilling a bone that includes an in-situ hardenable material or that can be infused with a hardenable material. The fill material also can include other “fillers” such as filaments, microspheres, powders, granular elements, flakes, chips, tubules and the like, autograft or allograft materials, as well as other chemicals, pharmacological agents or other bioactive agents.

“Flowable material” includes its ordinary meaning and is defined as a material continuum that is unable to withstand a static shear stress and responds with an irrecoverable flow (a fluid)—unlike an elastic material or elastomer that responds to shear stress with a recoverable deformation. Flowable material includes fill material or composites that include a fluid (first) component and an elastic or inelastic material (second) component that responds to stress with a flow, no matter the proportions of the first and second component, and wherein the above shear test does not apply to the second component alone.

“Substantially” or “substantial” mean largely but not entirely. For example, substantially may mean about 10% to about 99.999%, about 25% to about 99.999% or about 50% to about 99.999%. “Osteoplasty” includes its ordinary meaning and means any procedure wherein fill material is delivered into the interior of a bone.

“Vertebroplasty” includes its ordinary meaning and means any procedure wherein fill material is delivered into the interior of a vertebra.

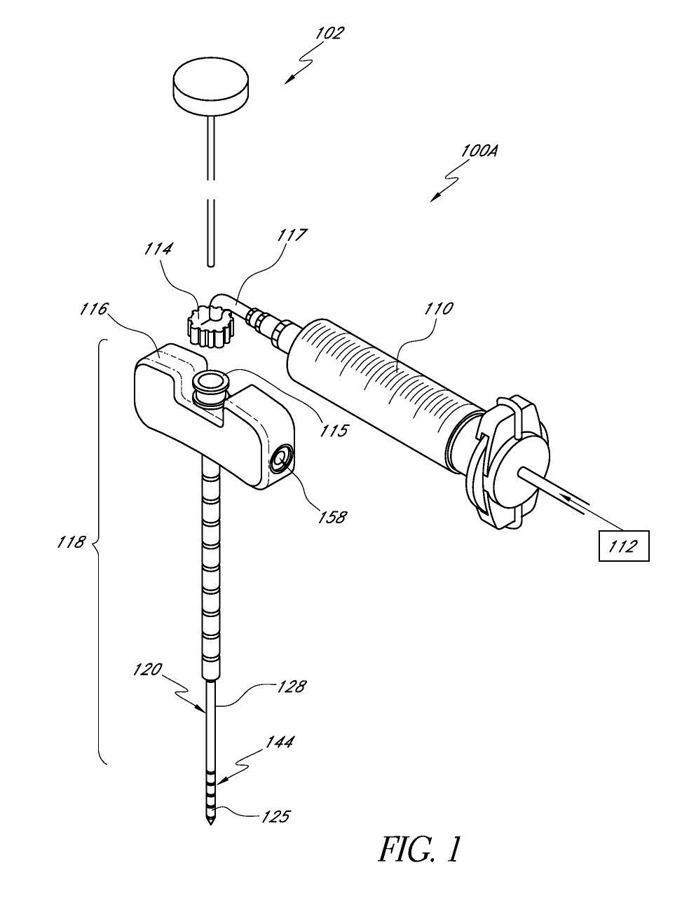

As background, a vertebroplasty procedure, according to one embodiment, would include inserting the introducer of FIG. 1 through a pedicle of a vertebra for accessing the osteoporotic cancellous bone. The initial aspects of the procedure are similar to a conventional percutaneous vertebroplasty wherein the patient is placed in a prone position on an operating table. The patient is typically under conscious sedation, although general anesthesia is an alternative. The physician injects a local anesthetic (e.g., 1% Lidocaine) into the region overlying the targeted pedicle or pedicles as well as the periosteum of the pedicle(s). Thereafter, the physician uses a scalpel to make a 1 to 5 mm skin incision over each targeted pedicle. Thereafter, the introducer is advanced through the pedicle into the anterior region of the vertebral body, which typically is the region of greatest compression and fracture. Prior to said advancement of the introducer, an elongated member 102 (see FIG. 1), such as rod or trocar, can be inserted into the channel in the introducer so as to block the channel and inhibit the introduction of debris into the channel of the introducer during advancement of the introducer into the vertebral body. The elongated member 102 can be withdrawn from the introducer when the treatment region has been reached. The physician can confirm the introducer path posterior to the pedicle, through the pedicle and within the vertebral body by anteroposterior and lateral X-Ray projection fluoroscopic views. The introduction of infill material as described below can be imaged several times, or continuously, during the treatment depending on the imaging method.

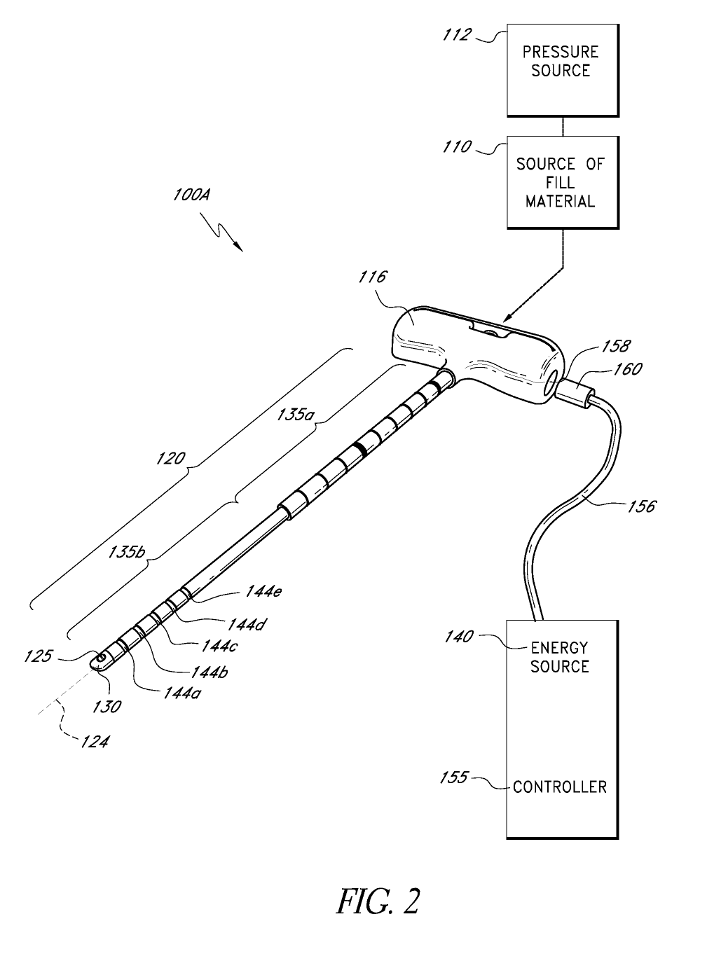

Referring to FIGS. 1-2, one embodiment of bone fill introducer or injector system 100A is shown that is configured for treatment of the spine in a vertebroplasty procedure. Introducer system 100A can be used for injecting a fill material from a source 110, wherein injection of the fill material can be carried out by a pressure mechanism or source 112. The pressure mechanism 112 can be a manually operated syringe loaded with bone fill material, a hydraulically actuated syringe or any other pressurized source of fill material. In one embodiment, the fill material source includes a sleeve with a piston therein that is drivable by a fluid pushed by the hydraulic source as pressure mechanism 112.

The source 110 of fill material can include a coupling or fitting 114 for sealably locking to a cooperating fitting 115 at a proximal end or handle 116 of an elongated bone fill injector 118. The injector 118 can have a introducer sleeve 120 with a proximal portion 135a and a distal portion 135b. In one embodiment, a syringe-type source 110 can be removably coupled directly to the fitting 115 in the handle 116 via a threaded coupling, a Luer lock or the like, the fitting 115 communicating with the introducer sleeve 120. In another embodiment, shown in FIG. 1, a flexible tube 117, can be used to couple the source 110 to the introducer 120. However, in other embodiments, the tube 117 can be a rigid tube or a bendable (deformable) tube.

In FIGS. 1-4, it can be seen that the bone fill injector 118 includes an elongated sleeve 120 with an interior channel 122 extending about an axis 124, wherein the channel 122 terminates in a distal outlet opening 125. In the illustrated embodiment, the outlet opening 125 is a single opening. However, in other embodiments, the outlet opening can be a plurality of openings disposed about the radially outward surface 128 or an opening at the distal tip 130 of the introducer sleeve 120. The distal tip 130 can be blunt or sharp.

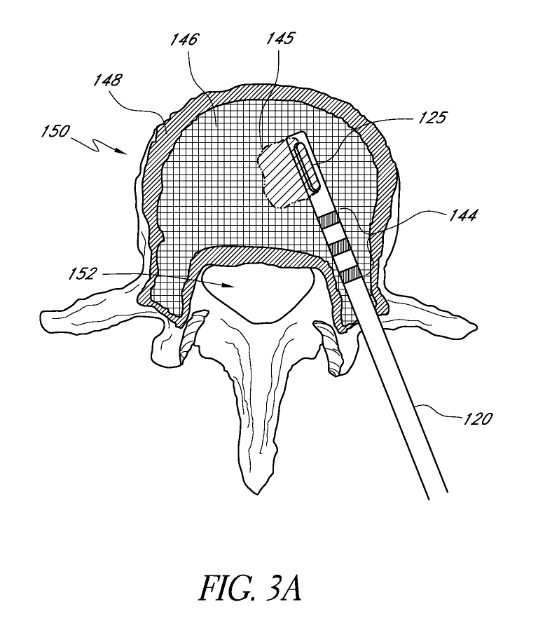

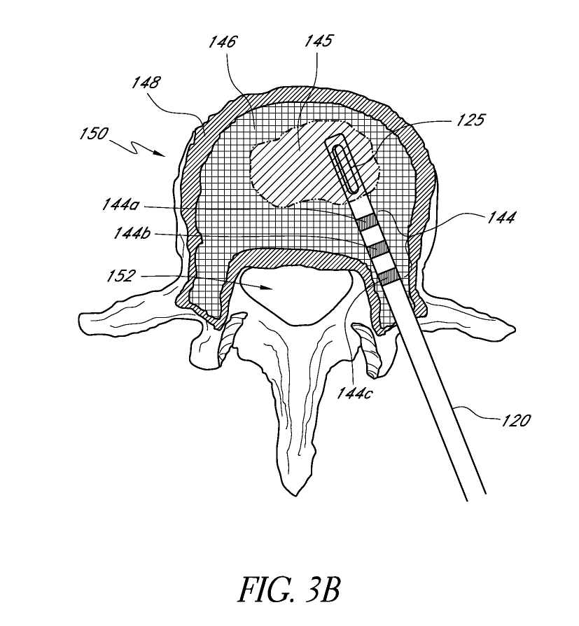

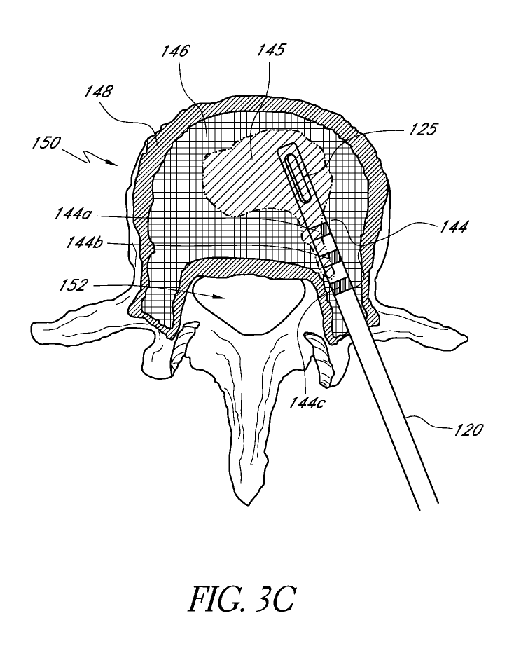

As can be seen in FIGS. 1-2, the exterior surface of introducer sleeve 120 carries at least one sensor system 144 that is adapted to sense the flow or movement of a fill material 145 (see FIGS. 3A-3C) proximate to the sensor system 144. In the illustrated embodiment, the sensor system 144 has a plurality of spaced apart electrodes 144a-144e, which can be ring-type electrodes. Though the sensor system 144 in the illustrated embodiment shows five ring-type electrodes, any number of electrodes, and other types of electrodes can also be used. The introducer sleeve 120 and sensor system 144 can be used to monitor and prevent extravasation of fill material 145 in a vertebroplasty procedure.

In one embodiment and method of use, referring to FIGS. 3A-3C, the introducer sleeve 120 can be used in a conventional vertebroplasty with a single pedicular access or a bi-pedicular access. The fill material 145 can be a bone cement, such as PMMA, that is injected into cancellous bone 146 which is within the interior of the cortical bone surface 148 of vertebra 150.

FIGS. 3A-3B illustrate a progressive flow of cement 145 provided through the outlet opening 125 of the introducer sleeve 120 into the interior of the vertebra 150. FIG. 3C depicts a situation that is known to occur where bone is fractured along the entry path of the introducer 120 or the pressurized cement 145 finds the path of least resistance to be a retrograde path along the surface of introducer 120. The retrograde flow of cement 145, as in FIG. 2C, if allowed to continue, could lead to cement extravasation into the spinal canal 152 which can lead to serious complications. As can be understood from FIG. 3C, the sensor system 144 is configured to be actuated when bone cement 145 comes proximate to, or into contact with, the sensor system 144.

In one embodiment, as shown in FIGS. 1-3C, the sensor system 144 can have a plurality of spaced apart electrodes (e.g., electrodes 144a, 144b, 144c) that are coupled to an electrical source 140 via, for example an electrical cable 156, to an electrical connector 158 in the proximal end of the introducer 118. As shown in FIG. 2, the electrical connector 158 can connect with a corresponding connector 160 coupled to the electrical source via the electrical cable 156.

The electrical source 140 can provide a low voltage direct current or Rf current between the opposing potentials of spaced apart electrodes (e.g., electrodes 144a, 144b, 144c). The voltage can be from about 0.1 volt to 500 volts, or from about 1 volt to 5 volts and can create a current path through the tissue between a pair of electrodes. The current can be continuous, intermittent and/or multiplexed between different electrode pairs or groups of electrodes.

In the illustrated embodiment, the arrangement of electrodes can be spaced apart ring-type electrodes and axially spaced apart as shown in FIGS. 1 and 2. However, in other embodiments the electrodes can be discrete elements, helically spaced electrodes, or the electrodes can be miniaturized electrodes as in thermocouples, MEMS devices or any combination thereof. The number of sensors or electrodes can range from about 1 to 100 and can be adapted to cooperate with a ground pad or other surface portion of the introducer sleeve 120.

In one embodiment, the electrodes can include a PTC or NTC material (positive temperature coefficient of resistance or negative temperature coefficient of resistance) to thereby function as a thermistor to allow for measurement of temperature, as well as functioning as a sensor. As shown in FIG. 2, a controller 155 can be electrically connected to the sensor system 144, which can measure at least one selected parameter of the current flow to determine a change in a parameter, such as impedance. When non-conductive bone cement 145 contacts one or more electrodes 144a-144e of the sensor system 144, the controller 155 can identify a change in the selected electrical parameter and generate a signal to the operator of the introducer system 100A. The scope of the invention includes sensor systems capable of sensing a change in electrical properties, reflectance, fluorescence, magnetic properties, chemical properties, mechanical properties or a combination thereof.

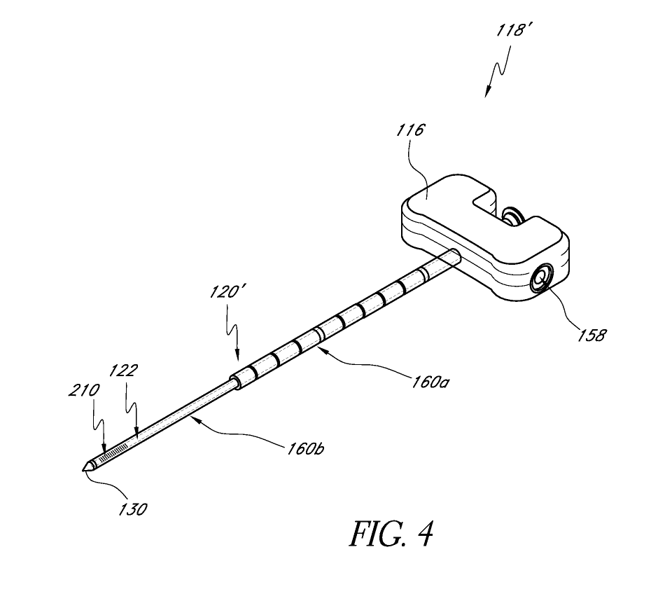

Now referring to FIG. 4, another view of an injector 118′ is shown, which includes an introducer 120′ having a proximal portion 160a that is larger in cross-section than a distal portion 160b thereof, with corresponding larger and smaller bore portions therein. This configuration allows for lesser injection pressures since the cement flow needs to travel less distance through the smallest diameter distal portion 160b of the introducer sleeve 120′. The distal portion 160b of the introducer can have a cross section ranging between about 2 mm and 4 mm with a length ranging between about 40 mm and 60 mm. The proximal portion 160a of the introducer can have a cross section ranging between about 5 mm and 15 mm., or between about 6 mm and 12 mm. However, other suitable cross-sectional dimensions and lengths can be used.

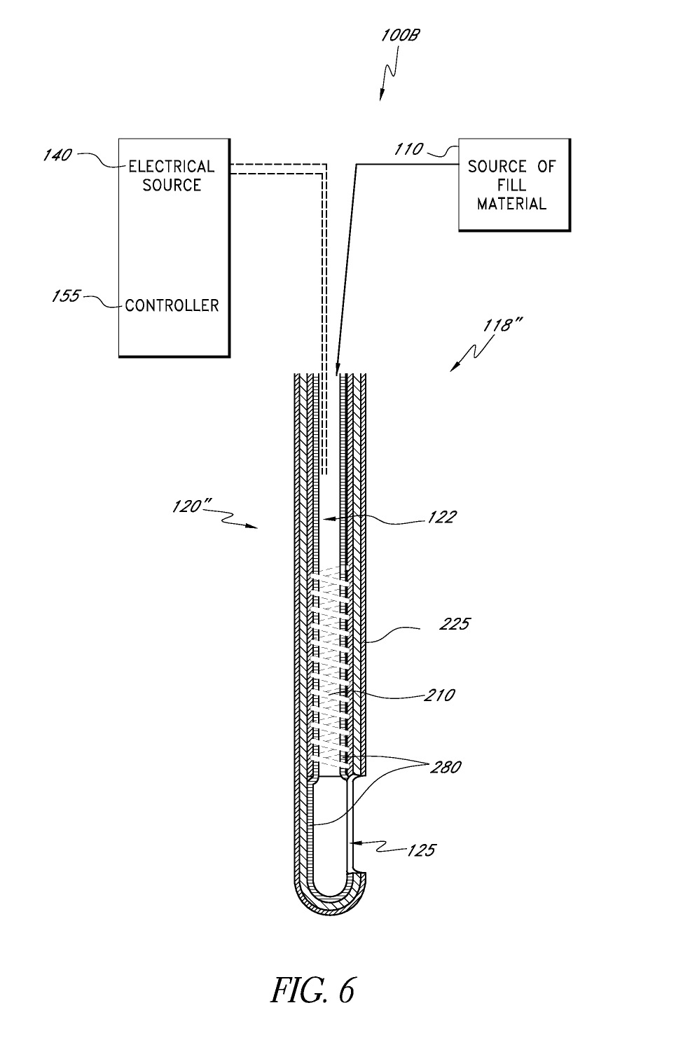

In the system of FIGS. 1-4, the bone fill injection system 100A can also include a thermal energy emitter 210 within the interior channel 122 of the introducer 120′ for heating a flow of bone cement prior to ejection of the same from the outlet opening 125 in the introducer sleeve 120′. In the illustrated embodiment, the thermal energy emitter 210 is disposed in the distal portion of the introducer 120′. However, in other embodiments, the thermal energy emitter 210 can be disposed in other locations within the introducer 120′. In one embodiment, the thermal energy emitter 210 is configured to raise the temperature of bone cement, for example chilled bone cement, to body temperature or within about 5° C. above or below body temperature. In another embodiment, thermal energy emitter 210 is configured to raise the temperature of chilled bone cement 145 to the range of 50° C. to 55° C. to accelerate polymerization and increase the viscosity of the bone cement, which can be a PMMA or similar bone cement. The thermal energy emitter 210 can be an Rf emitter adapted for heating a conductive bone cement as disclosed in the following co-pending U.S. patent applications: Ser. No. 11/165,652 filed Jun. 24, 2005; Ser. No. 11/165,651 filed Jun. 24, 2005; Ser. No. 11/208,448 filed Aug. 20, 2005; and Ser. No. 11/209,035 filed Aug. 22, 2005. In another embodiment, as shown in FIG. 6, the thermal energy emitter 210 can deliver thermal energy by conduction to bone cement flowing through the introducer, as further discussed below. The thermal energy emitter 210 can be selected from the group consisting of a resistively heated emitter, a light energy emitter, an inductive heating emitter, an ultrasound source, a microwave emitter and any other electromagnetic energy emitter to cooperate with the bone cement.

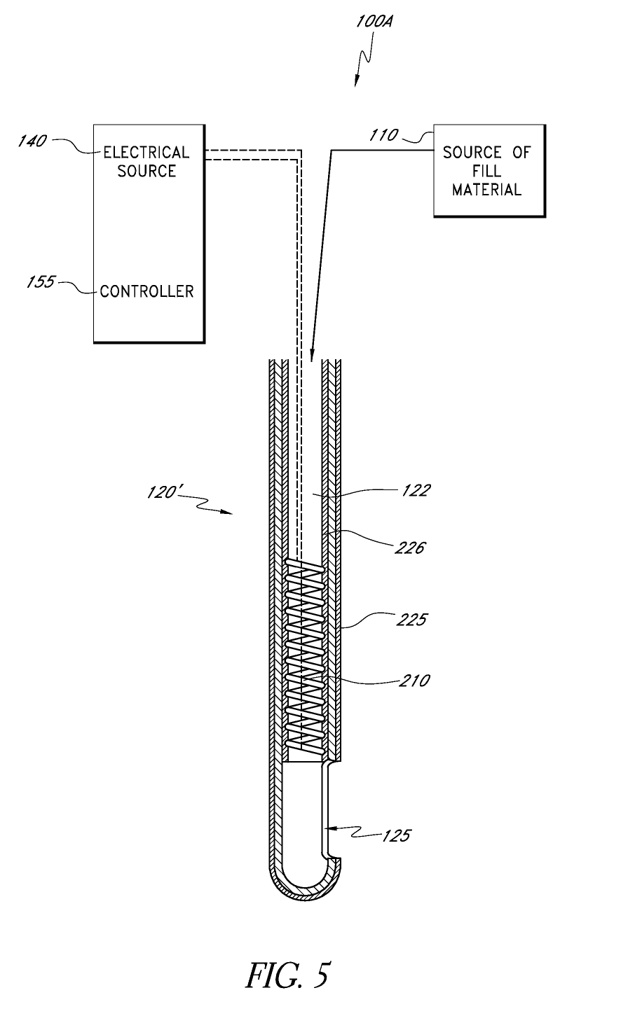

In the embodiment illustrated in FIGS. 4 and 5, the controller 155 is adapted to control all parameters of (i) heating the bone cement, (ii) the cement injection pressure and/or flow rate, (iii) energy delivery to cement flows in or proximate the distal end of the introducer and (iv) energy delivery to sense retrograde flows about the exterior surface of the introducer. In one embodiment depicted in FIG. 5, the thermal energy emitter 210 is a resistively heated element 210 in any suitable from (e.g., a helical configuration) that is provided within the interior channel 122 of the introducer 120′. The heating element 210 can further be formed from (or coated with) a positive temperature coefficient material and coupled to a suitable voltage source, such as the electrical source 140, to provide a constant temperature heater as is known in the art. In the illustrated embodiment, the heating element 210 is carried within an insulative coating 226 on an inner surface of the introducer 120′, where the introducer 120′ can be metal. As discussed above in connection with FIG. 2, the electrical source 140 can be coupled to the handle 116 via electrical connectors 158, 160.

With continued reference to the embodiment in FIG. 5, the exterior surface of the probe has a coating 225 that can include a thin layer of an insulative amorphous diamond-like carbon (DLC) or a diamond-like nanocomposite (DCN). It has been found that such coatings have high scratch resistance, lubricious and non-stick characteristics that are useful in the bone cement injectors disclosed herein that are configured for carrying electrical current for (i) impedance sensing purposes; (ii) for energy delivery to bone fill material; and/or (iii) ohmic heating of tissue. For example, when inserting a bone cement injector, such as the injector 118, through the cortical bone surface of a pedicle and then into the interior of a vertebra, it is important that the exterior insulative coating portions do not fracture, chip or scratch to thereby ensure that the electrical current carrying functions of the injector are not compromised.

The amorphous diamond-like carbon coatings and the diamond-like nanocomposites are available, for example, from Bekaert Progressive Composites Corporations, 2455 Ash Street, Vista, Calif. 92081 or its parent company or affiliates. Further information on said coatings can be found at: http://www.bekaert.com/bac/Products/Diamond-like%20coatings.htm, the contents of which are incorporated herein by reference. The diamond-like coatings comprise amorphous carbon-based coatings with high hardness and a low coefficient of friction. The amorphous carbon coatings exhibit non-stick characteristics and excellent wear resistance. The coatings are thin, chemically inert and have a very low surface roughness. In one embodiment, the coatings have a thickness ranging between 0.001 mm and 0.010 mm; or between 0.002 mm and 0.005 mm. The diamond-like carbon coatings are a composite of sp2 and sp3 bonded carbon atoms with a hydrogen concentration between 0 and 80%. Another suitable diamond-like nanocomposite coating (a-C:H/a-Si:O; DLN) is made by Bekaert and is suitable for use in the bone cement injector described in the embodiments above. The materials and coatings are known by the names Dylyn®Plus, Dylyn®/DLC and Cavidur®.

FIG. 6 illustrates another embodiment of a bone fill introducer system 100B. The introducer system 100B is similar to the introducer system 100A discussed above in connection with FIG. 5. Thus, the reference numerals used to designate corresponding components in the introducer system 100A and the introducer system 100B are identical, except that a “″” is added where there are differences.

The introducer system 100B has a bone cement injector 118″ that again includes a thermal energy emitter 210 within the interior passageway 122 of an introducer 120″ for heating a flow of bone cement before the same is ejected from the outlet opening 125 of the introducer 120″. In this embodiment, the thermal energy emitter 210 is a helical coil that is resistively heated. A lubricious surface 280 is provided in the interior passageway 122 of the introducer 120″. In one embodiment, the lubricious surface 280 can be provided via a coating applied to the inner surface of the interior channel 122. In another embodiment, the introducer sleeve 120″ can be made of the lubricious material. In still another embodiment, the lubricious surface 280 can be provided via a sleeve fitted into the introducer sleeve 120″. The lubricious surface 280 advantageously facilitates the flow of bone fill material 145 through the interior passageway 122 of the introducer 120″.

In one embodiment, the lubricious surface 280 can be a fluorinated polymer surface, such as Teflon® or polytetrafluroethylene (PTFE). Other suitable fluoropolymer resins are Fluorinated ethylenepropylene (FEP) and Perfluoroalkoxy (PFA). Other materials also can be used such as FEP (Fluorinated ethylenepropylene), ECTFE (Ethylenechlorotrifluoroethylene), Ethylene Tetrafluoroethylene (ETFE), Polyethylene, Polyamide, Polyvinylidene Difluoride (PVDF), Polyvinyl chloride and silicone.

In one embodiment, the surface 280 of the flow channel 122 has a static coefficient of friction of less than 0.5, less than 0.2, or less than 0.1.

In another embodiment, at least a portion of the surface 280 of the flow channel 122 is ultrahydrophobic or hydrophobic which may better prevent a hydrophilic cement from sticking to the surface 280 as it flows through the channel 122.

In still another embodiment, at least a portion of the surface 280 of the flow channel 122 is hydrophilic, which may prevent a hydrophobic cement from sticking.

In yet another embodiment, the surface 280 of the flow channel 122 has high dielectric strength, a low dissipation factor, or a high surface resistivity.

In another embodiment, the surface 280 of the flow channel 122 is oleophobic.

In another embodiment, the surface 280 of the flow channel 122 has a wetting contact angle greater than 70°, greater than 85°, and greater than 100°.

In another embodiment, the surface 280 of the flow channel 122 has an adhesive energy of less than 100 dynes/cm, less than 75 dynes/cm, and less than 50 dynes/cm.

In another embodiment, the surface 280 of the flow channel 122 includes a low coefficient of friction polymer or ceramic.

The system 100A, 100B can use any suitable energy source, other that radiofrequency energy, to accomplish the purpose of altering the viscosity of the fill material 145. The energy source for altering fill material can be, for example, at least one of a radiofrequency source, a laser source, a microwave source, a magnetic source and an ultrasound source. In one embodiment, each of these energy sources can preferably deliver energy to a cooperating, energy sensitive filler component carried by the fill material. For example, such filler can be suitable chromophores for cooperating with a light source, ferromagnetic materials for cooperating with magnetic inductive heating means, or fluids that thermally respond to microwave energy.

In one embodiment, the apparatus disclosed above can also be configured with a thermal energy emitter that comprises at least in part an electrically conductive polymeric layer. In such an apparatus, the electrically conductive polymeric layer has a positive temperature coefficient of resistance.

The scope of the invention includes using additional filler materials such as porous scaffold elements and materials for allowing or accelerating bone ingrowth. In one embodiment, the filler material can comprise reticulated or porous elements of the types disclosed in co-pending U.S. patent application Ser. No. 11/146,891, filed Jun. 7, 2005, titled “Implants and Methods for Treating Bone,” which is incorporated herein by reference in its entirety and should be considered a part of this specification. Such fillers also can carry bioactive agents. Additional fillers, or the conductive filler, also can include thermally insulative solid or hollow microspheres of a glass or other material for reducing heat transfer to bone from the exothermic reaction in a typical bone cement component.

The above description of the invention is intended to be illustrative and not exhaustive. Particular characteristics, features, dimensions and the like that are presented in dependent claims can be combined and fall within the scope of the invention. The invention also encompasses embodiments as if dependent claims were alternatively written in a multiple dependent claim format with reference to other independent claims. Specific characteristics and features of the invention and its method are described in relation to some figures and not in others, and this is for convenience only. While the principles of the invention have been made clear in the above descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

Of course, the foregoing description is that of certain features, aspects and advantages of the present invention, to which various changes and modifications can be made without departing from the spirit and scope of the present invention. Moreover, the bone treatment systems and methods need not feature all of the objects, advantages, features and aspects discussed above. Thus, for example, those skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed bone treatment systems and methods.

What is claimed is:

1. An apparatus for delivering a bone fill material to a vertebra, comprising:
an injector configured for introduction into a vertebral body, at least a portion of the injector positionable within the vertebral body, the injector having a lubricious surface layer that defines a flow channel extending through the injector to at least one outlet opening; and
a thermal energy emitter operably coupled to the introducer, at least a portion of the surface layer disposed between the thermal energy emitter and the flow channel, the thermal energy emitter configured to apply energy to the bone fill material flowing through the flow channel via conduction through the surface layer.

2. The apparatus of claim 1, wherein the surface layer has a static coefficient of friction of less than 0.5.

3. The apparatus of claim 1, wherein the surface layer has a static coefficient of friction of less than 0.1.

4. The apparatus of claim 1, wherein the surface layer comprises a ceramic material.

5. The apparatus of claim 1, wherein the surface layer comprises a polymer.

6. The apparatus of claim 5, wherein the surface layer comprises a material selected from the group consisting of PTFE (Polytetrafluoroethylene), PFA (Perfluoroalkoxy), FEP (Fluorinatedethylenepropylene), ECTFE (Ethylenechlorotrifluoroethylene), ETFE (Ethylene Tetrafluoroethylene), Polyethylene, Polyamide, PVDF (Polyvinylidene Difluoride), Polyvinyl chloride and silicone.

7. The apparatus of claim 1, wherein the thermal energy emitter comprises at least one conductor coupled to an electrical source.

8. The apparatus of claim 7, wherein the at least one conductor comprises a resistive heating element.

9. The apparatus of claim 7, wherein the at least one conductor comprises a material having a positive temperature coefficient of resistance.

10. The apparatus of claim 1, further comprising at least one electrode on an outer surface of the injector, the electrode configured to sense a flow of bone fill material proximate the electrode.

11. The apparatus of claim 10, wherein the at least one electrode comprises a positive temperature coefficient of resistance, the electrode coupleable to an energy source and configured to apply energy to at least one of tissue and bone fill material proximate the electrode.

12. The apparatus of claim 1, wherein the surface layer comprises a material having a positive temperature coefficient of resistance.

13. An apparatus for delivering a bone cement to a bone, comprising a bone cement injector having a flow channel extending therethrough to at least one outlet opening in a distal end of the injector, wherein a surface of the flow channel comprises a polymeric layer.

14. The apparatus of claim 13, further comprising a thermal energy emitter disposed in the flow channel, the thermal energy emitter coupleable to an electrical source and configured to deliver energy to bone cement flowing through the flow channel.

15. The apparatus of claim 14, wherein the thermal energy emitter is embedded in the polymeric layer, the thermal energy emitter configured to deliver energy to the bone cement via conduction through the polymeric layer.

16. The apparatus of claim 14, wherein the thermal energy emitter comprises at least in part an electrically conductive polymeric layer.

17. The apparatus of claim 16, wherein the electrically conductive polymeric layer has a positive temperature coefficient of resistance.

18. The apparatus of claim 13, wherein the polymeric layer comprises a material selected from the group consisting of PTFE (Polytetrafluoroethylene), PFA (Perfluoroalkoxy), FEP (Fluorinatedethylenepropylene), ECTFE (Ethylenechlorotrifluoroethylene), ETFE (Ethylene Tetrafluoroethylene), Polyethylene, Polyamide, PVDF (Polyvinylidene Difluoride), Polyvinyl chloride and silicone.

19. The apparatus of claim 13, wherein the polymeric layer comprises a static coefficient of friction of less than 0.5.

20. The apparatus of claim 13, wherein at least a portion of the surface of the flow channel is hydrophobic so as to inhibit hydrophilic bone cement from adhering to said surface.

21. The apparatus of claim 13, wherein at least a portion of the surface of the flow channel is hydrophilic so as to inhibit hydrophobic cement from adhering to said surface.

22. The apparatus of claim 13, wherein the surface of the flow channel is oleophobic.

23. An apparatus for delivering a bone cement to a bone, comprising a bone cement injector having a flow channel extending therethrough to at least one outlet opening in a distal end of the injector, wherein a surface of the flow channel comprises a ceramic layer.

24. The apparatus of claim 23, further comprising a thermal energy emitter disposed in the flow channel, the thermal energy emitter coupleable to an electrical source and configured to deliver energy to bone cement flowing through the flow channel.

25. The apparatus of claim 23, wherein the surface of the flow channel comprises a wetting contact angle greater than 70°.

26. The apparatus of claim 25, wherein the surface of the flow channel has a wetting contact angle greater than 100°.

27. The apparatus of claim 23, wherein the surface of the flow channel has an adhesive energy of less than 100 dynes/cm.

28. The apparatus of claim 27, wherein the surface of the flow channel has an adhesive energy of less than 50 dynes/cm.

29. The apparatus of claim 23, wherein the surface of the flow channel comprises a static coefficient of friction of less than 0.5.
